(19) United States
(12) Patent Application Publication
(10) Pub. No.: US 2020/0273574 A1
(43) Pub. Date: Aug. 27, 2020

(54) METHODS FOR ANALYZING METABOLIC DRIFT IN A SUBJECT

(71) Applicants: Ronan BOUTIN, REZE (FR); Arnaud LECLEVE, Paris (FR)

(72) Inventor: Ronan BOUTIN, REZE (FR)

(21) Appl. No.: 16/629,247

(22) PCT Filed: Jul. 6, 2018
(86) PCT No.: PCT/EP2018/068430

§ 371 Date: Jan. 7, 2020

(30) Foreign Application Priority Data
Jul. 7, 2017 (FR) 1756464

(51) Int. Cl. G16H 50/20; G06F 17/18

(52) U.S. Cl. CPC G16H 50/20; G06F 17/18

(57) ABSTRACT

The present invention relates to a method for analyzing the metabolic drift of at least one quantitative biological parameter in a subject and thereby make it possible to provide a method for evaluating the drift state of a subject and/or helping diagnose an illness or evaluate a subject's risk of suffering from an illness.

The present invention also provides a method for defining an average reference value and a reference standard deviation of a biological parameter which are appropriate for analyzing the metabolic drift of said biological parameter and a method for optimizing a cohort of subjects in order to study a biological parameter.

### TECHNICAL FIELD

The present invention concerns methods for analysing biological parameter(s) of a subject.

### STATE OF THE ART

The analysis of one or more biological parameters in a subject is an indispensable tool to assist the diagnosis of a pathology. There exist more or less direct links between some biological parameters and given pathologies, for example between glycaemia and diabetes, iron level and anaemia, blood pressure and hypertension, the presence of a pathogen and an infection related to this pathogen, sedimentation rate and an infection or inflammation, etc. . . . . The measurement of some biological parameters thereby allows the orienting or confirmation of a diagnosis. It allows the monitoring of patient response to treatment by tracking the changes in a parameter related to the disease concerned.

The analysis of a biological parameter generally consists of measuring the value of this biological parameter, and determining whether the value obtained lies within a range of reference values defined by a minimum reference threshold value and maximum reference threshold value. The results of biological analysis are therefore classified into three categories: (i) outside the minimum reference threshold value (ii) between the minimum and maximum reference threshold values or (iii) outside the maximum reference threshold value. A value lying within a range of reference values will be considered to be «normal». The «normality» of a value lying outside the range of reference values is left to the appreciation of the practitioner, in particular in relation to the difference between this value and the reference threshold values.

Reference values can vary as a function of the geographical origin, gender and/or age of the subject and optionally as a function of the assay method used. In general, reference values correspond to the results obtained in a reference population in which the individuals are free of any pathology and/or treatment likely to modify the measured values. In practice, reference threshold values defined by the medical profession are not necessarily indexed on statistical analyses of the population.

Reference values are the subject of international recommendations, and in particular by the International Federation of Clinical Chemistry and Laboratory Medicine (IFCC-LM) and the Clinical and Laboratory Standards Institute (CLSI).

There still remains a need to provide biological analysis methods that are more specific and/or allow earlier detection of one or more biological parameters having values corresponding to an abnormal or potentially abnormal state of health.

### DESCRIPTION

The inventor has surprisingly evidenced that a biological parameter can show drift or potential drift i.e. corresponding to an abnormal or potentially abnormal state of health for this biological parameter, even if the value of this biological parameter lies within a range of reference values for this biological parameter.

In particular, in the present invention, a biological parameter which does not vary over time around a mean reference value is considered to show drift or potential drift even if its value lies within a range of reference values. With the present invention it is therefore possible to detect biological parameters corresponding to an abnormal or potentially abnormal clinical condition, but which would not have been considered as such when comparing their values with a range of reference values.

Analysis of the drift of a biological parameter according to the invention can advantageously be used to assist early diagnosis of a disease e.g. by directing towards diagnosis of a disease in its asymptomatic phase.

Analysis of the drift of a biological parameter according to the invention also advantageously allows the preventing or at least delaying of the onset of a disease, for example by allowing the setting-up of early treatment to halt or slow the drifting of said biological parameter, for example even before the onset of the first symptoms.

Advantageously, the present invention also allows medical analysis to be oriented towards measurement of at least one another biological parameter statistically related to a biological parameter that has been identified as showing drift or potential drift. For example, said other biological parameter can be directly correlated with a pathology which would not first come to mind for a practitioner on the basis of clinical examination and/or biological analysis not taking metabolic drift into consideration.

A first subject of the invention concerns a method for analysing the metabolic drift of at least one quantitative biological parameter in a subject, characterized in that it comprises the following steps:
a) providing the values of at least one quantitative biological parameter, measured at least at two different spaced-apart times t1 and t2 to obtain at least two values v1 and v2;
b) determining whether said values fluctuate around a mean reference value for said quantitative biological parameter;
c) if said values do not fluctuate around said mean reference value, concluding therefrom that said biological parameter shows drift or potential drift.

The quantitative parameter measured at step a) is preferably selected from the group consisting of a serum parameter, an infection parameter, a clinical parameter and a multi-parameter indicator.

The above analysis method is preferably characterized in that:
said quantitative biological parameter shows drift if:
the absolute value of the difference between (i) the mean of said values and (ii) the mean reference value is greater than or equal to 1 reference standard deviation; or
the absolute value of the difference between (i) the mean of said values and (ii) the mean reference value is greater than or equal to 0.5 reference standard deviation and less than 1 reference standard deviation, and the absolute value of the difference between (i) at least one of said values and (ii) the mean reference value is greater than or equal to 2 reference standard deviations; or
at least one of said values is greater than or equal to a maximum reference threshold value and/or at least one of said values is lower than or equal to a minimum reference threshold value;
and/or
said quantitative biological parameter shows potential drift if:
the absolute value of the difference between (i) at least one of said values or the mean of said values and (ii) the mean reference value is greater than or equal to 0.5 reference standard deviation and less than 1 reference standard deviation; and
if the absolute value of the difference between (i) each of said values and (ii) the mean reference value is less than 2 reference standard deviations.

When said quantitative biological parameter shows drift or potential drift, the method such as defined above preferably comprises a subsequent step to measure the value of said quantitative biological parameter at another time tᵢ.

When the quantitative biological parameter shows drift or potential drift, the method such as defined above preferably comprises the following steps: measuring the slope of a line of best fit passing through the values provided at step a) and deducing therefrom whether the parameter showing drift or potential drift is stable, worsening or improving.

When said quantitative biological parameter shows drift or potential drift, the method such as defined above preferably comprises at least one of the following subsequent steps:
providing the value of at least one other biological parameter statistically related to the biological parameter showing drift or potential drift; and/or
implementing a method to diagnose a disease or risk of suffering from a disease, said disease being associated with said biological parameter showing drift or potential drift.

The mean reference value and/or reference standard deviation is preferably defined using the method for defining an optimised mean reference value and optimised reference standard deviation of a biological parameter X such as defined below.

A further subject of the invention concerns a method for defining an optimised mean reference value and optimised reference standard deviation of a biological parameter X, comprising:
a) providing the value of the biological parameter X measured at a given time and/or values of the biological parameter X measured at different spaced-apart times and the value of at least one other biological parameter measured at a given time and/or values of at least one other biological parameter measured at different spaced-apart times, for each subject in a group of at least 50 subjects;
b) identifying the biological parameter(s) statistically related to biological parameter X on the basis of said value and/or of the mean of said values of biological parameter X and of at least one other biological parameter;
c) for each biological parameter identified at step b), removing from the group:
   (i) those subjects in whom this biological parameter lies outside a reference threshold value and/or in whom this biological parameter shows drift or potential drift, if it is a quantitative biological parameter; and/or
   (ii) those subjects in whom this biological parameter is influential if it is a qualitative biological parameter, to define a sub-group of subjects;
c′) optionally, providing the values of at least one serum biological parameter measured at least at two different spaced-apart times t1 and t2 for each subject in the sub-group defined at step c) and removing from the sub-group those subjects in whom this serum biological parameter lies outside a reference threshold value and/or in whom this biological parameter shows drift or potential drift, to define a second sub-group of subjects; and
d) defining the mean and standard deviation of biological parameter X in the sub-group of subjects defined at step c) or in the second sub-group of subjects defined at step c′).

A further subject of the invention concerns a method to evaluate the state of drift in a subject and/or to assist diagnosis of a disease or evaluation of a subject's risk of suffering from a disease, characterized in that it comprises the implementation of the method for analysing metabolic drift in a subject such as defined above.

A further subject of the invention concerns a method for optimising a cohort of subjects for the purpose of studying a biological parameter X, comprising the steps of:
a) providing the value at a given time and/or a mean of values at different spaced-apart times of the biological parameter X and of at least one biological parameter statistically related to biological parameter X for each subject in a cohort of subjects;
b) for each biological parameter, removing from the cohort or placing in a sub-group:
   (i) those subjects in whom this biological parameter lies outside a reference threshold value and/or in whom this biological parameter shows drift or potential drift, if it is a quantitative biological parameter; and/or
   (ii) those subjects in whom this biological parameter is influential, if it is a qualitative biological parameter,
the reference threshold value being greater than or equal to the sum of the mean reference value and one reference standard deviation, or being less than or equal to the difference between the mean reference value and one reference standard deviation, to obtain an optimised cohort.

A further subject of the invention concerns a method to evaluate the state of drift of a subject and/or to assist diagnosis of a disease or evaluation of a subject's risk of suffering from a disease, comprising:
a) measuring the value of at least one biological parameter at least at one given time t1 in a biological sample of the subject, to obtain at least one value v1;
b) comparing the value(s) obtained at step a) with a reference threshold value and/or determining whether the values obtained at step a) fluctuate around a mean reference value; and
c) if the value or one of the values of said biological parameter is higher than a maximum reference threshold value or lower than a minimum reference threshold value and/or if said values do not fluctuate around a mean reference value, providing the value of at least one other biological parameter statistically related to said biological parameter and/or deducing therefrom whether or not the subject is suffering from or likely to suffer from the disease.

For example, said biological parameter can be the mean platelet volume, said biological parameter being statistically related to free thyroxine and said disease being hypothyroidism and/or a Graves-Basedow disease.

A further subject of the invention concerns a computer programme comprising programme code instructions to execute one or more steps of the methods such as defined above, when said programme is computer-executed.

Biological Parameter

By «biological parameter» it is designated herein any quantitative or qualitative parameter allowing direct or indirect evaluation of a subject's state of health.

The biological parameter is preferably selected from the group consisting of a serum parameter, an infection parameter, a genetic parameter, a non-serum parameter (e.g. a clinical parameter or lifestyle-related parameter) and a multi-parameter indicator.

The qualitative parameter can be a qualitative parameter that remains fixed over time (e.g. gender or the presence of a given gene) or it may vary over time.

Genetic and lifestyle-related parameters are preferably qualitative parameters.

Serum, clinical, non-serum and infection parameters can be quantitative or qualitative parameters.

The biological parameter, and in particular the serum, infection, genetic parameters and some non-serum parameters can be measured in a biological sample of a subject.

The biological sample can be a blood sample, urine sample, cerebrospinal fluid sample, stool sample or a biopsy.

The biological sample may have been subjected to one or more treatment steps prior to analysis (e.g. purification, centrifugation, filtration, precipitation, PCR and/or RT-PCT).

By «serum parameter» it is designated herein a biological parameter measured in a blood sample or a sample obtained from a blood sample (e.g. in plasma).

For example, the serum parameter can be selected from the group consisting of:
the number, concentration and/or proportion of blood cells or populations of given blood cells: for example the number, concentration and/or proportion of erythrocytes (also called red blood cells), leukocytes (also called white blood cells), granulocytes, poly-neutrophils (also called polymorphonuclear neutrophils or neutrophil granulocytes), poly-eosinophils (also called polynuclear eosinophils), poly-basophils (also called polynuclear basophils), monocytes, lymphocytes or platelets;
complete blood count (CBC);
haemoglobin concentration;
haematocrit, i.e. the volume occupied by the red blood cells in a given volume of whole blood;
thrombocyte count;
sedimentation rate;
MCV (Mean corpuscular volume);
MCHC (Mean corpuscular haemoglobin concentration);
MCH (Mean corpuscular haemoglobin);
the quantity, concentration and/or proportion of a given enzyme e.g. lipasemia, the quantity, concentration and/or proportion of Alkaline Phosphatase, GGT, ASAT transaminase or ALAT transaminase;
the quantity, concentration and/or proportion of lipids, in particular total cholesterol, HDL cholesterol, LDL cholesterol or triglycerides,
glycaemia;
the quantity, concentration and/or proportion of chlorine, uric acid, urea, or creatinine;
electrophoresis of proteins, in particular the quantity, concentration and/or proportion of total proteins, albumin, alpha 1 globulin, alpha 2 globulin, beta 1 globulin, beta 2 globulin and/or gamma globulin;
the protein profile, in particular the quantity, concentration and/or proportion of serum proteins such as IgA, IgG, IgM and/or IgE;
electrolyte panel, in particular the quantity, concentration and/or proportion of one or more ions such as sodium, potassium, calcium, magnesium, chlorine, and/or bicarbonate, e.g. in blood plasma;
the quantity, concentration and/or proportion of one or more hormones;
the quantity, concentration and/or proportion of an antigen e.g. free PSA (prostate-specific antigen) and/or conjugated PSA; or
the blood group (e.g. the blood group ABO).

The infection parameter is related to the presence or absence of a pathogen in a subject.

The quantity, concentration and/or proportion of the pathogen can be of importance in determining whether the subject is suffering from an infection with said pathogen.

If the infection parameter is qualitative, it is given the value for example of «infection», «no infection» or «possible infection» with a given pathogen. The pathogen can be selected from the group consisting of a virus, a bacterium, a fungus, a protozoa and a parasite.

For example, the virus is a virus of mononucleosis, a cytomegalovirus, an herpes virus or HIV.

For example, the bacterium is Helicobacter (preferably Helicobacter pylori).

For example, the fungus is Candida (preferably Candida albicans, Candida dubliniensis, Candida glabrata, Candida guilliermondii, Candida kefyr, Candida krusei, Candida lusitaniae, Candida parapsilosis and/or Candida tropicalis).

By “genetic parameter” it is designated herein a qualitative parameter related to the genome of the subject, related for example to the presence of a given gene or given group of genes, a mutation, a variant and/or a given polymorphism.

For example, the genetic parameter is selected from the group consisting of the HLA group, mutation of the HFE gene causing hemochromatosis and mutation of the CFTR gene causing cystic fibrosis.

The non-serum parameter herein designates any parameter which is not a serum, infection or genetic parameter.

Preferably, the non-serum parameter is a clinical parameter or lifestyle-related parameter.

By «clinical parameter» it is particularly designated herein a quantitative or qualitative parameter which is not measured in a biological sample.

For example, the clinical parameter is selected from the group consisting of blood pressure (e.g. systolic pressure and/or diastolic pressure), height, weight, age, waist size, gender (in particular male or female), season of birth, pathology (e.g. a cancer, diabetes, anaemia, depression).

For example, the lifestyle-related parameter is selected from the group consisting of cigarette smoking, food diet (e.g. gluten-free, dairy-free, vegetarian and/or vegan), sedentary lifestyle, place of abode (e.g. north or south of France), hours of sunshine and day or night working hours.

A multi-parameter indicator is the result of a mathematical equation comprising at least one quantitative biological parameter as variable, for example at least two quantitative biological parameters. For example, the multi-parameter indicator may comprise or consist of a product, ratio, sum, subtraction, mean and/or an equation involving cos, sin, tan and/or exp, and notably involving at least one quantitative biological parameter.

The multi-parameter indicator is therefore obtained from one or more values of at least one quantitative biological parameter which can be measured in particular in a biological sample of a subject.

Non-limiting examples of a multi-parameter marker are:
the number of white blood cells divided by the number of red blood cells;
the quantity or concentration of HDL cholesterol divided by the quantity or concentration of LDL cholesterol;
the quantity or concentration of free PSA (prostate-specific antigen) divided by the quantity or concentration of conjugated PSA; or
the number of lymphocytes divided by the number of polymorphonuclear neutrophils.

By «biological parameter X statistically related to a biological parameter Y» it is designated herein two biological parameters which are significantly statistically related.

For example, two quantitative variables are statistically related if they tend to vary one in relation to the other.

Statistical significance between two biological parameters can be demonstrated by any statistical method well known to persons skilled in the art, e.g. linear, polynomial, logarithmic or non-linear regression, variance analysis (ANOVA), Student's test, chi-square test, cross tabulation, General Linear Model and/or Partial Least Squares.

To assess whether or not two quantitative biological parameters are statistically related, the statistical method can be linear, polynomial, logarithmic or non-linear regression for example.

To assess whether or not a quantitative biological parameter and a qualitative biological parameter are statistically related the statistical method can for example be an ANOVA method or General Linear Model.

To assess whether or not two qualitative biological parameters are statistically related, the statistical method can for example be a cross tabulation or chi-square statistical method.

Preferably, two biological parameters are statistically related if the level of statistical significance is higher than or equal to 90%, preferably higher than or equal to 95%, more preferably higher than or equal to 99.5%, for example higher than or equal to 99.9%. Statistical significance corresponds to the probability that the result obtained is not accidental.

Subject

The term «subject» designates herein a human subject or a non-human animal subject.

A non-human animal subject is preferably a mammal e.g. a cat, dog, rodent, primate, equine, bovine, ovine, caprine animal.

Alternatively, the non-human animal subject is not a mammal.

Preferably, the subject is a human subject, also called an «individual».

The human subject can be of any gender e.g. a male or female of any age for example an infant, toddler, young child, adolescent, adult or elderly person.

The subject can be a healthy subject, appear to be a healthy subject, show at least one symptom of a disease, have at least one biological parameter not included within a reference range of values for this parameter and/or likely to be suffering from a disease.

Mean value, standard deviation, threshold value and reference range

By «mean reference value» it is preferably meant the mean of the values of a given biological parameter in a reference population.

Alternatively, the mean reference value can be a fixed value e.g. by an authority, this value not necessarily corresponding to the mean of the values of a given biological parameter in a reference population.

By «reference standard deviation», it is designated herein the square root of the variance in the values of a given biological parameter in a reference population.

By «reference population» also called «reference group», it is designated a population of individuals in which the biological parameter of interest is measured.

In one particular embodiment, the reference population can be formed of healthy individuals for example i.e. showing a good general state of health.

The reference population preferably comprises at least 50 subjects, preferably at least 60 subjects, more preferably at least 70 subjects, at least 80 subjects, at least 90 subjects, further preferably at least 100 subjects, at least 110 subjects, for example at least 120 subjects.

By «reference threshold value», it is designated herein a reference value corresponding to a tolerance threshold. For example, a biological parameter having a value higher than or equal to a maximum reference threshold value or a value lower than a minimum reference threshold value will be considered to be out-of-tolerance. The reference threshold values define limits beyond which medical action must normally be taken.

Reference threshold values are generally defied in relation to the mean reference value. In one advantageous embodiment, the minimum reference threshold value can be equal to: −[n×reference standard deviation] and/or the maximum reference threshold value can be equal to: +[n×reference standard deviation]. For example, the minimum reference threshold value can be equal to: −2 reference standard deviations and/or the maximum reference threshold value can be equal to: +2 reference standard deviations. They may also be more or less arbitrary values e.g. defined by an authority for example further to strong professional agreements.

A reference threshold value equaling + or −2 standard deviations generally relates to a warning threshold corresponding to a condition that is probably pathological i.e. beyond which a value is considered to correspond to a condition that is probably pathological.

By «range of reference values», also called «reference range», it is designated the range between the minimum reference threshold value and maximum reference threshold value, these values being contained within the range.

Method for Analysing the Metabolic Drift of at Least One Biological Parameter in a Subject

A first subject of the invention is therefore a method for analysing the metabolic drift of at least one biological parameter in a subject.

By the expression «metabolic drift» or by the term «drift» associated with a biological parameter it is meant herein the fact that the values of this biological parameter measured over time do not fluctuate around a mean reference value.

For a so-called «normal» biological parameter, the values fluctuate over time around a mean reference value i.e. the mean of the values of this biological parameter measured over time is equal or equivalent to the mean reference value, said mean reference value preferably being an optimised mean reference value, in particular obtained with the method such as defined below. Once the values of the biological parameter measured over time do not fluctuate around a mean reference value, the fluctuations are no longer chance fluctuations but translate drift or potential drift of the biological parameter.

A mean of values that is considered equivalent to the mean reference value is for example strictly higher than [«mean reference value»−0.5×«reference standard deviation»] and strictly lower than [«mean reference value»+0.5×«reference standard deviation»].

For example, if the mean reference value equals 10 and the reference standard deviation is 0.2, then a value differing from 10 of 9.9 to 10.1 is equivalent to the mean reference value.

The terms «biological parameter» and «subject» are in particular such as defined above in the sections under this heading.

A particular subject of the present invention is a method for analysing the metabolic drift of at least one quantitative biological parameter in a subject, characterized in that it comprises the following steps:
a) providing the values of at least one quantitative biological parameter measured at least at two different spaced-apart times t1 and t2, to obtain at least two values v1 and v2;
b) determining whether said values fluctuate around a mean reference value for said quantitative biological parameter;
c) if said values do not fluctuate around said mean reference value, concluding therefrom that said biological parameter shows drift or potential drift.

The method for analysing metabolic drift is preferably implemented by a computer or at least partly implemented by a computer.

The method for analysing metabolic drift is preferably an in vitro and/or ex vivo method.

At step a), the values are provided of at least one quantitative biological parameter measured at least at two different spaced-apart times t1 and t2 to obtain at least two values v1 and v2.

The method for analysing metabolic drift such as defined above therefore may or may not comprise a prior step to measure said quantitative biological parameter in a biological sample, in particular at least at two different times t1 and t2. The measurements performed over time are therefore made on biological samples of the same subject taken at different times.

The biological samples, in which the biological parameter is measured over time, are preferably of same type and/or are preferably taken and/or measured under the same conditions.

In one advantageous embodiment, at step a) the values are provided of at least one quantitative biological parameter measured at least at three different spaced-apart times t1, t2 and t3 to obtain at least three values v1, v2 and v3, for example at least at four times t1, t2, t3 and t4, to obtain at least four values v1, v2, v3 and v4.

More generally, step a) may entail providing the values of at least one quantitative biological parameter measured at i spaced-apart times t1 to ti, to obtain i values v1 to vi, i being a positive integer higher than or equal to 2.

By «spaced-apart times» or «different times», it is meant that the measurements of the biological parameter are spaced out over time, preferably by at least 24 h. Measurement frequency is particularly dependent on the biological parameter under consideration.

For example, the biological parameter can be measured on average every week, every two weeks, every three weeks, every month, every two months, every three months, every six months or even once a year.

The quantitative biological parameter measured at step a) is preferably selected from the group consisting of a serum parameter, an infection parameter, a clinical parameter and a multi-parameter indictor. These different parameters are particularly such as defined above under the section «biological parameter».

At step b), it is determined whether said values fluctuate around a mean reference value for said quantitative biological parameter.

In particular, the mean reference value is such as defined above.

In one preferred embodiment, the mean reference value is obtained with the method such as defined below to define an optimised mean reference value and an optimised reference standard deviation of a biological parameter X, suitable for analysing metabolic drift.

The fluctuation of values around a mean reference value can be visually evaluated, for example by means of a graph showing the measured values for said biological parameter as a function of time. If the values obtained have random or substantially random distribution around the mean reference value, in particular having values alternately above and below the mean reference value, said values fluctuate around the mean reference value.

On the contrary, if said values are systematically higher or mostly higher than the mean reference value, or conversely systematically lower or mostly lower than the mean reference value, said values do not fluctuate around the reference mean.

Alternatively, the fluctuation of the values around a mean reference value can be evaluated by comparing:
the absolute value of the difference between (i) the mean of said values and (ii) the mean reference value; and/or
the absolute value of the difference between (i) at least one of said values, preferably each of said values, and (ii) the mean reference value,
   with at least one value equal to [n×reference standard deviation], n being a positive integer higher than or equal to 0.25, preferably higher than or equal to 0.5, for example equal to 0.5 or 1 or 1.5 or 2.

To finetune analysis of drift, the fluctuation of values around a mean reference value can be evaluated by comparing:
the difference between (i) the mean of said values, and (ii) the mean reference value; and/or
the difference between (i) at least one of said values, preferably each of said values, and (ii) the mean reference value,
   with at least one value equal to [n×reference standard deviation] and/or with at least one value equal to −[n×reference standard deviation], n being a positive integer higher than or equal to 0.25, preferably higher than or equal to 0.5, for example equal to 0.5, or 1 or 1.5 or 2.

One preferred value of the invention equal to [n×reference standard deviation] is equal to 0.5 reference standard deviation, 1 reference standard deviation, 1.5 reference standard deviations or 2 reference standard deviations.

The mean reference value and the reference standard deviation are particularly such as defined above under the section «Mean value, standard deviation, threshold value and reference range».

In one preferred embodiment of the invention, the mean reference value and the reference standard deviation are therefore optimised values for analysis of drift, obtained for example with the method such as defined below to define a mean reference value and reference standard deviation of a biological parameter X, suitable for analysing metabolic drift.

At step c), said biological parameter is considered to show drift or potential drift if said values do not fluctuate around said mean reference value.

If the measured values fluctuate around said mean reference value for one or more periods and do not fluctuate around said mean reference value for one or more periods, the drift state of the biological parameter is given as a function of time. In other words, it is indicated whether said biological parameter is or is not considered to show drift or potential drift, for each period of time during which fluctuation differs.

The drift state is in particular such as defined below.

As explained below, it is also possible to distinguish sub-drift states and hence to indicate at step c) the different successive drift states and/or sub-drift states of the biological parameter over time.

In one embodiment of the invention,
if the absolute value of the difference between (i) the mean of said values and (ii) the mean reference value is higher than or equal to [n×reference standard deviation]; and/or
if the absolute value of the difference between (i) at least one of said values, preferably each of said values, and (ii) the mean reference value is higher than or equal to [n×reference standard deviation],
   it is deduced therefrom that said values do not fluctuate around said mean reference value, in particular if n is higher than or equal to 0.5.

In one advantageous embodiment of the invention,
if the difference between (i) the mean of said values and (ii) the mean reference value is greater than or equal to [n×reference standard deviation] or less than or equal to −[n×reference standard deviation]; and/or
if the difference between (i) at least one of said values, preferably each of said values, and (ii) the mean reference value, is greater than or equal to [n×reference standard deviation] or less than or equal to −[n×reference standard deviation],
   it is deduced therefrom that said values do not fluctuate around said mean reference value, in particular if n is higher than or equal to 0.5.

In one preferred embodiment, the method such as defined above is characterized in that:
said quantitative biological parameter shows drift if:
   the absolute value of the difference between (i) the mean of said values and (ii) the mean reference value is higher than or equal to 1 reference standard deviation; or
   the absolute value of the difference between (i) the mean of said values and (ii) the mean reference value is higher than or equal to 0.5 reference standard deviation and lower than 1 reference standard deviation, and the absolute value of the difference between (i) at least one of said values and (ii) the mean reference value is higher than or equal to 2 reference standard deviations; or
   at least one of said values is higher than or equal to a maximum reference threshold value and/or at least one of said values is lower than a minimum reference threshold value;
   and/or
said quantitative biological parameter shows potential drift if:
   the absolute value of the difference between (i) at least one of said values or the mean of said values and (ii) the mean reference value is higher than or equal to 0.5 reference standard deviation and lower than 1 reference standard deviation, and
   if the absolute value of the difference between (i) each of said values and (ii) the mean reference value is lower than 2 reference standard deviations.

The minimum or maximum reference threshold value is particularly such as defined above under the section «Mean value, standard deviation, threshold value and reference range».

In one advantageous embodiment of the invention, the method such as defined above is characterized in that, when said quantitative biological parameter shows drift or potential drift, the method such as defined above comprises a subsequent step to measure the value of said quantitative biological parameter at another time tᵢ.

This step can allow confirmation for example of potential drift and/or the monitoring of changes in a parameter showing drift or potential drift, in particular after setting up treatment and/or during treatment intended to correct said biological parameter i.e. intended to halt the drift or potential drift of said biological parameter.

The other time tᵢ is preferably at least one week, preferably at least two weeks, more preferably at least three weeks, for example three weeks or four weeks after the last measured value. The other time tᵢ can also be one month, two months, three months, six months afterwards.

Preferably, at least for as long as the biological parameter shows drift or potential drift, the biological parameter is regularly measured over time, for example on average every week, every two weeks, every three weeks, every month, every two months, every three months, every 6 months or even once a year.

In one advantageous embodiment of the invention, the method such as defined above is characterized in that, when the quantitative biological parameter shows drift or potential drift, the method such as defined above comprises the following steps: measuring the slope of a line of best fit passing through the values provided at step a) and deducing therefrom whether the parameter showing drift or potential drift is stable, worsening or improving.

The parameter showing drift or potential drift is worsening for example if the slope of the line of best fit is such that the values obtained over time move away from the mean reference value.

The parameter showing drift or potential drift is stable for example if the slope of the line of best fit is zero.

The parameter showing drift or potential drift is improving for example if the slope of the line of best fit is such that the values obtained over time move close to the mean reference value.

The line of best fit is obtained with any suitable method well known to skilled persons. The line of best fit is preferably obtained with a linear regression model e.g. affine fitting, least square method, maximum likelihood and/or Bayesian inference.

It is possible to define different states or sub-states of drift as a function of the values obtained. It will be obvious for persons skilled in the art that the invention is in no way limited to states and/or sub-states of drift or potential drift which are given here as examples.

For example, it is possible to distinguish between the following states: no drift, drift and potential drift.

The state corresponding to no drift for example can comprise the sub-states «excellent» or «good».

A drift state for example can comprise the sub-states «drift» and «major drift».

The state or sub-state of drift or potential drift can be qualified as «low» if the mean of the values is lower than the mean reference value and «high» if the mean of the values is higher than the mean reference value.

For example, a biological parameter can be classified as having excellent status if the absolute value of the difference between (i) the mean of the values and (ii) the mean reference value is strictly lower than 0.5 standard deviation, and if the absolute value of the difference between (i) the most recent value and (ii) the mean reference value is strictly lower than 0.5 standard deviation.

For example, a biological parameter can be classified as having good status when:
the absolute value of the difference between (i) the mean of the values and (ii) the mean reference value is strictly lower than 0.5 standard deviation, and the difference between (i) the most recent value and (ii) the mean reference value is greater than or equal to 0.5 standard deviation;
the absolute value of the difference between (i) the mean of the values and (ii) the mean reference value is strictly lower than 0.5 standard deviation, and the difference between (i) the most recent value and (ii) the mean reference value is less than or equal to −0.5 standard deviation;
the difference between (i) the mean of the values and (ii) the mean reference value is from 0.5 standard deviation to 1 standard deviation, and the difference between (i) the most recent value and (ii) the mean reference value is strictly less than 0.5 standard deviation;
the difference between (i) the mean of the values and (ii) the mean reference value is from −1 standard deviation to −0.5 standard deviation, and the difference between (i) the most recent value and (ii) the mean reference value is strictly greater than −0.5 standard deviation;
the difference between (i) the mean of the values and (ii) the mean reference value is from 0.5 standard deviation to 1 standard deviation, and the difference between (i) at least one of the values and (ii) the mean reference value is strictly greater than 0.5 standard deviation; or
the absolute value of the difference between (i) the mean of the values and (ii) the mean reference value is from −1 standard deviation to −0.5 standard deviation, and the difference between (i) at least one of the values and (ii) the mean reference value is strictly less than −0.5 standard deviation.

A biological parameter is classified as showing potential drift for example when:
the difference between (i) the mean of the values and (ii) the mean reference value is greater than or equal to 0.5 reference standard deviation and is less than 1 reference standard deviation, and the difference between (i) each of the values and (ii) the mean reference value is greater than or equal to 0.5 reference standard deviation and less than 1 reference standard deviation;
the difference between (i) the mean of the values and (ii) the mean reference value is greater than or equal to −1 reference standard deviation and is less than −0.5 reference standard deviation, and the difference between (i) each of the values and (ii) the mean reference value is greater than or equal to −1 reference standard deviation and is less than −0.5 reference standard deviation;
the difference between (i) the mean of the values and (ii) the mean reference value is greater than or equal to 1 reference standard deviation and is less than 2 reference standard deviations, and the difference between (i) the most recent value and (ii) the mean reference value is less than 1 reference standard deviation; or
the difference between (i) the mean of the values and (ii) the mean reference value is greater than or equal to −2 reference standard deviations and is less than −1 reference standard deviation, and the difference between (i) the most recent value and (ii) the mean reference value is greater than −1 reference standard deviation.

A biological parameter is classified as showing drift for example when:
the difference between (i) the mean of the values and (ii) the mean reference value is greater than or equal to 1 reference standard deviation and is less than 2 reference standard deviations;
the difference between (i) the mean of the values and (ii) the mean reference value is greater than or equal to −2 reference standard deviations and is less than −1 reference standard deviation;
the difference between (i) the mean of the values and (ii) the mean reference value is greater than or equal to 1 reference standard deviation and is less than 2 reference standard deviations, and the difference between (i) each of the values and (ii) the mean reference value is greater than or equal to 1 reference standard deviation and is less than 2 reference standard deviations;
the difference between (i) the mean of the values and (ii) the mean reference value is greater than or equal to −2 reference standard deviations and is less than −1 reference standard deviation, and the difference between (i) each of the values and (ii) the mean reference value is greater than or equal to −2 reference standard deviations and is less than −1 reference standard deviation;
the difference between (i) the mean of the values and (ii) the mean reference value is greater than or equal to 1 reference standard deviation and is less than 2 reference standard deviations, and the difference between (i) the most recent value and (ii) the mean reference value is greater than 2 reference standard deviations;
the difference between (i) the mean of the values and (ii) the mean reference value is greater than or equal to −2 reference standard deviations and is less than −1 reference standard deviation, and the difference between (i) the most recent value and (ii) the mean reference value is less than −2 reference standard deviations;
the difference between (i) the mean of the values and (ii) the mean reference value is greater than or equal to 2 reference standard deviations and is less than 3 reference standard deviations, and the difference between (i) the most recent value and (ii) the mean reference value is less than 2 reference standard deviations; or
the difference between (i) the mean of the values and (ii) the mean reference value is greater than or equal to −3 reference standard deviations and is less than −2 reference standard deviations, and the difference between (i) the most recent value and (ii) the mean reference value is greater than −2 reference standard deviations.

A biological parameter is classified for example as showing major drift when:
the difference between (i) the mean of the values and (ii) the mean reference value is greater than or equal to 2 reference standard deviations, for example greater than or equal to 3 reference standard deviations;
the difference between (i) the mean of the values and (ii) the mean reference value is less than or equal to −2 reference standard deviations, for examples less than or equal to −3 reference standard deviations; or
the most recent value is lower than a minimum reference threshold value or higher than a maximum reference threshold value.

When at least 3 values are provided at step a), step b) of the method such as defined above preferably comprises analysis of the drift of the values taken two-by-two, starting with the oldest measured value or alternatively with the most recent measured value.

The method such as defined above can then be characterized in that:
at step b), it is determined whether said values fluctuate around a mean reference value for said quantitative biological parameter for each period of time between two times tx and tz such that the drift state between each of the successive values taken two-by-two between time tx and tz is the same (i.e. the drift state between vx and vx+1, the state between vx+1 and vx+2, the state between vx+2 and vx+3, etc. up until the state vx+y−1 and vz, with x+y=z, are the same);
at step c), for each period of time defined at step b), it is concluded that said biological parameter shows drift or potential drift if said values do not fluctuate around said mean reference value.

Step b) may advantageously comprise comparison of the drift state and/or drift sub-state of values vi and vi+1 with the drift state and/or drift sub-state of values vi+1 and vi+2:
if the drift states and/or drift sub-states are the same, step b) may then comprise comparison of the drift state and/or drift sub-state of values vi, vi+1 and vi+2 with the drift state and/or drift sub-state of values vi+2 and vi+3, and so forth;
if the drift states and/or drift sub-states differ, step b) may then comprise comparison of the drift state and/or drift sub-state of values vi+2 and vi+3 with the drift state and/or drift sub-state of values vi+3 and vi+4, and so forth, and step c) will comprise at least two different drift states or sub-states of the different biological parameter including a drift state or drift sub-state defined between times vi and vi+1.

At step c), several different drift states and/or drift sub-states of the biological parameter over time can therefore be determined.

For example, step b) may comprise:
(i) determining the drift state and/or drift sub-state of values v1 and v2;
(ii) determining the drift state of values v2 and v3;
(iii) if the drift state and/or drift sub-state is the same for values v1 and v2 and for values v2 and v3:
   a. determining the drift state and/or drift sub-state of values v1, v 2 and v3,
   b. determining the drift state and/or drift sub-state of values v3 and v4,
   c. if the drift state and/or drift sub-state is the same for values v1, v2 and v3 and for values v3 and v4, determining the drift state and/or drift sub-state of values v1 to v4 and comparison thereof with the drift state and/or drift sub-state of values v4 and v5, and so forth,
   d. if the drift state and/or drift sub-state for values v1, v2 and v3 differs from that for values v3 and v4, inferring therefrom a drift state and/or drift sub-state between times t1 and t3 at step c) and comparing the drift state and/or drift sub-state of values v4 and v5 with that of values v5 and v6, etc.,
(iv) if the drift state and/or drift sub-state for values v1 and v2 differs from that for values v2 and v3:
   a. inferring therefrom the drift state and/or drift sub-state of the biological parameter between times t1 and t2 from values v1 and v2,
   b. comparing the drift state and/or drift sub-state of values v3 and v4 with that of values v4 and v5, etc. . . . .

The analysed values taken two-by-two are each preferably measured within an interval of less than 5 years, for example within an interval of less than two years.

The values analysed two-by-two preferably lie within less than 2 standard deviations.

If the values analysed two-by-two differ by a least 2 standard deviations, each of these values is analysed individually or with another value.

When the quantitative biological parameter shows drift or potential drift, the method such as defined above may comprise a subsequent step to provide the value of at least one other biological parameter statistically related to the biological parameter showing drift or potential drift. Preferably, the method may then comprise analysis of the metabolic drift of said at least one other biological parameter statistically related to the biological parameter showing drift or potential drift, for example with the analysis method such as defined above. Alternatively, the value of said other biological parameter is compared with a mean reference value and/or reference threshold value.

Said other biological parameter can be a quantitative parameter (e.g. a serum parameter, infection parameter, clinical parameter or multi-parameter indicator) or a qualitative parameter (e.g. a genetic parameter, infection parameter, non-serum parameter or lifestyle-related parameter).

A biological parameter statistically related to a biological parameter is as defined in particular in the section «Biological parameter».

Nonlimiting examples are given below.

Vitamin D is an example of biological parameter statistically related to Vitamin B12.

The inventor has evidenced that when the concentration of vitamin B12 shows low drift, more than 61% of patients suffer from vitamin D deficiency, whereas when the concentration of vitamin B12 does not show low drift, only 44% of patients suffer from vitamin D deficiency.

Therefore, if the concentration of vitamin B12 shows low drift, in particular when the concentration of vitamin B12 is lower than 316 pg/ml, the method such as defined above preferably comprises a subsequent step to measure the concentration of vitamin D.

If the concentration of vitamin D shows drift, in particular with a vitamin D concentration of less than 20 ng/ml, which corresponds to vitamin D deficiency, the method preferably comprises a subsequent step to determine the HLA group. If the patient's group is HLA-DQ02, HLA-DQ8, HLA-DR3 or HLA-A1, the method preferably comprises a step to evaluate intestinal absorption or to diagnosis celiac disease. For example, a level lower than 21 ng/ml is more particularly specific to the population carrying the genes HLA-DQ02, HLA-DQ8, HLA-DR3 or HLA-A1, these same genes being known to cause intestinal malabsorption in particular malabsorption of vitamin D.

For example, if the mean platelet volume shows low drift, the method preferably comprises a subsequent step to measure free thyroxine.

When the quantitative biological parameter and/or a biological parameter statistically related to said biological parameter shows drift or potential drift, the method such as defined above may comprise a subsequent step to implement a method, preferably in vitro and/or ex vivo, to diagnose a disease or risk of suffering from a disease, said disease being associated with the biological parameter showing drift or potential drift and/or with the biological parameter statistically related to said biological parameter showing drift or potential drift.

The method of the invention has the advantage of allowing earlier diagnosis of a disease. Diagnosis can effectively be made as soon as a biological parameter associated with this disease shows drift or potential drift, instead of waiting until this biological parameter becomes out-of-tolerance.

In one advantageous embodiment of the invention, when said quantitative biological parameter shows drift or potential drift, the method such as defined above comprises at least one of the following subsequent steps:
providing the value of at least one other biological parameter statistically related to the biological parameter showing drift or potential drift, for example such as defined above; and/or
implementing a method, preferably in vitro and/or ex vivo, to diagnose a disease or risk of suffering from a disease, said disease being associated with the biological parameter showing drift or potential drift, or with said biological parameter statistically related to the biological parameter showing drift of potential drift, for example such as defined above.

For example, if the biological parameter showing drift or potential drift is the multi-parameter indicator defined by the number of white blood cells divided by the number of red blood cells, the method preferably comprises a step to diagnose or evaluate the risk of suffering from infection with Candida albicans, e.g. invasive candidiasis.

For example, if the biological parameter showing drift or potential drift is the multi-parameter indicator defined by the number of lymphocytes divided by the number of polymorphonuclear neutrophils, the method preferably comprises a step to diagnose or evaluate the risk of suffering from a Cytomegalovirus (CMV) infection.

Method for Defining an Optimised Mean Reference Value and Optimised Reference Standard Deviation of a Biological Parameter X, Suitable for Analysis of Metabolic Drift of Said Biological Parameter

The present invention also allows the providing of an optimised mean reference value and optimised reference standard deviation of a biological parameter X, for analysis of metabolic drift.

The method for defining a mean reference value and reference standard deviation of a biological parameter X according to the invention takes into account the possible drift or potential drift of biological parameter(s) statistically related to biological parameter X, to define an optimised group and to obtain a mean reference value and reference standard deviation that are more representative of optimum metabolic balance.

One subject of the invention is therefore a method for defining an optimised mean reference value and optimised reference standard deviation of a biological parameter X, that are suitable in particular for analysing metabolic drift of said biological parameter, comprising:
a) providing:
   the value of biological parameter X measured at a given time and/or values of the biological parameter X measured at different spaced-apart times; and
   the value of at least one other biological parameter measured at a given time and/or values of at least one other biological parameter measured at different spaced-apart times;
   for each subject in a group of at least 50 subjects;
b) identifying the biological parameter(s) statistically related to biological parameter X from said value and/or from the mean of said values of biological parameter X and of at least one other biological parameter;
c) for each biological parameter identified at step b), removing from the group:
   (i) those subjects in whom this biological parameter lies outside a reference threshold value and/or in whom this biological parameter shows drift or potential drift, if it is a quantitative biological parameter; and/or
   (ii) those subjects in whom this biological parameter is influential, if it is a qualitative biological parameter, to define a sub-group of subjects;
c′) optionally, providing the values of at least one serum biological parameter measured at two spaced-apart times t1 and t2 for each subject in the sub-group defined at step c) and removing from the sub-group those subjects in whom this serum biological parameter lies outside a reference threshold value and/or in whom this biological parameter shows drift or potential drift, to define a second sub-group of subjects; and
d) defining the mean and the standard deviation of biological parameter X in the sub-group of subjects defined at step c) or in the second sub-group of subjects defined at step c′), optionally taking into account at least one quantitative biological parameter.

A particular subject of the invention is a method for defining an optimised mean reference value and optimised reference standard deviation of a quantitative biological parameter X, comprising:
a) providing:
   a value of the quantitative biological parameter X measured at a given time or at least two values of the quantitative biological parameter X measured at different spaced-apart times;
   at least two values of at least one other quantitative biological parameter measured at different spaced-apart times; and
   optionally, the value of at least one other qualitative biological parameter measured at a given time and/or values of at least one other qualitative biological parameter measured at different spaced-apart times,
for each subject in a group of at least 50 subjects;
b) identifying the quantitative biological parameter(s), and optionally the qualitative biological parameter(s), statistically related to the quantitative biological parameter X, from one of said values and/or from the mean of said values of the quantitative biological parameter X, from one of said values or from the mean of said values of at least one other quantitative biological parameter, and optionally from the said value(s) of at least one other qualitative biological parameter;
c) for each biological parameter identified at step b), removing from the group: (i) those subjects in whom this quantitative biological parameter shows drift or potential drift and (ii) those subjects, if any, in whom this qualitative biological parameter is influential, to define a sub-group of subjects;
c′) optionally, providing at least two values of at least one serum biological parameter measured at different spaced-apart times for each subject in the sub-group defined at step c) and removing from the sub-group those subjects in whom this serum biological parameter shows drift or potential drift, to define a second sub-group of subjects; and
d) defining the mean and the standard deviation of biological parameter X in the sub-group of subjects defined a step c) or in the second sub-group of subjects defined a step c′).

The biological parameter is such as defined above under the section carrying the same heading.

The biological parameter is preferably selected from the group consisting of a serum parameter, infection parameter, clinical parameter, genetic parameter, lifestyle-related parameter and a multi-parameter indicator, each of these parameters particularly being such as defined above under the section «Biological parameter».

The biological parameter X is preferably a quantitative parameter.

The expression «biological parameter outside a reference threshold value» means that at least one of the values and/or the mean of the values of said biological parameter is higher than or equal to a maximum reference threshold value or is lower than or equal to a minimum reference threshold value.

The method for defining a mean reference value and reference standard deviation for a biological parameter X is preferably implemented by a computer or at least partly computer-implemented.

The method for defining a mean reference value and reference standard deviation of a biological parameter X is preferably an in vitro and/or ex vivo method.

At step a) there is provided:
the or one value of biological parameter X measured at a given time and/or values of biological parameter X e.g. at least two values measured at different spaced-apart times; and
the value of at least one other biological parameter measured at a given time and/or values of at least one other biological parameter measured at different spaced-apart times, e.g. at least two values, for each subject in a group of at least 50 subjects.

Step a) preferably comprises providing at least two values of at least one other quantitative biological parameter measured at different spaced-apart times and optionally the value of at least one other qualitative biological parameter measured at a given time and/or values of at least one other qualitative biological parameter measured at different spaced-apart times.

Therefore, the method for defining a mean reference value and reference standard deviation of a biological parameter X such as defined above may or may not comprise a prior step to measure said biological parameter X and at least one other biological parameter at a given time or at different spaced-apart times e.g. in a biological sample.

The measurements taken over time are performed on biological samples of the same subject taken at different times.

The biological samples on which the given biological parameter is measured over time are preferably of same type and/or are preferably taken and/or measured under the same conditions.

By «at spaced-apart times», it is meant that the measurements of the biological parameter are spread over time preferably by at least by 24 h. The frequency of measurements is dependent in particular on the biological parameter under consideration.

For example, the biological parameter can be measured on average every week, every two weeks, every three weeks, every month, every two months, every three months, every six months or once a year.

At step b) the biological parameter(s) statistically related to biological parameter X are identified from said value and/or from the mean of said values of biological parameter X and of at least one other biological parameter.

In particular, at step b) the quantitative biological parameter(s), and optionally the qualitative biological parameter(s), statistically related to the quantitative biological parameter X are identified from one of said values and/or from the mean of said values of the quantitative biological parameter X, from one of said values or from the mean of said values of at least one other quantitative biological parameter and optionally from one of said values of at least one other qualitative biological parameter.

A biological parameter statistically related to a biological parameter is in particular such as defined under the section «Biological parameter».

In particular, the statistical significance between two biological parameters can be shown using any statistical method well known to skilled persons, e.g. linear correlation, covariance, variance analysis (ANOVA), Student's test, chi-square test, cross tabulation.

Preferably, two biological parameters are statistically related if the level of statistical significance is greater than or equal to 90%, preferably greater than or equal to 95%, more preferably greater than or equal to 99%, for example greater than or equal to 99.5%.

At step c), for each biological parameter statistically related to the biological parameter X identified at step b), the following are removed from the group:
(i) those subjects in whom this biological parameter lies outside a reference threshold value and/or in whom this biological parameter shows drift or potential drift, if it is a quantitative biological parameter; and/or
(ii) the subjects in whom this biological parameter is influential, if it is a qualitative biological parameter, to define a sub-group of subjects.

In particular, at step c), for each biological parameter identified at step b), the following are removed from the group:
(i) those subjects in whom this quantitative biological parameter shows drift or potential drift; and
(ii) optionally those subjects in whom this qualitative biological parameter is influential, to define a sub-group of subjects.

Evaluation of the drift or potential drift of a biological parameter is particularly carried out as described above in the method for analysing the metabolic drift of at least one biological parameter in a subject.

Therefore, the method of the invention advantageously allows the removal from the group of those subjects in whom a biological parameter correlated with the biological parameter being examined shows drift or potential drift (by means of (i)). The method of the invention thereby allows optimisation of the reference group used to calculate the mean reference value and the reference standard deviation of biological parameter X.

By «subjects in whom this biological parameter is influential», it is meant subjects in whom this qualitative biological parameter has a value that it is not desired to take into consideration to determine the optimised reference mean and optimised reference standard deviation.

Nonlimiting examples of parameters which may be influential are gender, weight, height and/or blood group.

If a qualitative biological parameter is influential, the method such as defined above may comprise the defining of the mean reference value and reference standard deviation in the other sub-group or one of the other sub-groups corresponding to another value or another range of values of the qualitative parameter, in particular among the subjects removed at step c) ii). Optional step c′) and step d) can then be performed in each sub-group corresponding to another value or another range of values of the qualitative parameter.

For example, if gender is an influential biological parameter, the mean reference value and reference standard deviation can be respectively calculated in a sub-group comprising male subjects and in a sub-group comprising female subjects.

For example, blood group is a biological parameter having an influence on lipasemia values. Subjects of blood group A therefore globally lower lipasemia values than subjects of group O, and therefore significantly differing mean values and standard deviations. The optimised mean reference value and optimised reference standard deviation can then be respectively calculated in a sub-group formed of subjects of group A and in a sub-group formed of subjects of group O.

The method such as defined above may optionally comprise a step c′) at which the values are provided of at least one serum biological parameter measured at different times e.g. at least at two different spaced-apart times t1 and t2, for each subject of the sub-group(s) defined at step c), and at which those subjects in whom this serum biological parameter lies outside a reference threshold value and/or in whom this biological parameter shows drift or potential drift are removed from the sub-group(s), to define one or more second sub-groups of subjects.

Step c′) for example can allow the removal from the sub-group of one or more subjects who are in the course of developing an infection, deficiency or any other imbalance but not yet exhibiting symptoms thereof, the measured serum biological parameter preferably not being statistically related to biological parameter X.

Step c′) allows further optimisation of the reference group used to calculate the mean reference value and reference standard deviation of biological parameter X.

At step d) the mean and the standard deviation are defined of biological parameter X in the sub-group of subjects defined at step c) or in the second sub-group(s) of subjects defined at step c′), optionally taking into account at least one quantitative biological parameter.

Depending on the case, the optimised mean and optimised reference standard deviation can be defined as a function of one or more quantitative parameters statistically related to biological parameter X and/or as a function of one or more qualitative parameters statistically related to biological parameter X.

Nonlimiting examples are the mean and standard deviation of ideal weight defined as a function of the height of the subject, or the mean and standard deviation of haemoglobin concentration as a function of the height or weight of the subject.

Method for Optimising a Cohort of Subjects

The present invention also allows a method to be provided for optimising a cohort of subjects for the purpose of a study, for example on factors involved in and/or impacting a disease or response to a treatment.

The method for optimising a cohort of subjects according to the invention takes into consideration any drift or potential drift of biological parameter(s) statistically related to biological parameter X that is the subject of the study to define an optimised cohort.

One subject of the present invention is therefore a method for optimising a cohort of subjects for the purpose of a study on a biological parameter X, comprising the steps of:
a) providing the value at a given time, at least two values measured at different spaced-apart times and/or a mean of values at different spaced-apart times of biological parameter X and of at least one biological parameter statistically related to biological parameter X for each subject in a cohort of subjects;
b) for each biological parameter, removing from the cohort or placing in a sub-group:
   (i) those subjects in whom this biological parameter lies outside a reference threshold value and/or in whom this biological parameter shows drift or potential drift, if it is a quantitative biological parameter; and/or
   (ii) those subjects in whom this biological parameter is influential if it is a qualitative biological parameter,
   the reference threshold value being higher than or equal to the sum of the mean reference value and one reference standard deviation, or lower than or equal to the difference between the mean reference value and one reference standard deviation,
   to obtain one or more optimised cohorts.

A further subject of the invention is a method for optimising a cohort of subjects for the purpose of studying a biological parameter X, comprising the steps of:
a) providing (i) at least two values measured at different spaced-apart times and/or a mean of at least two values measured at different spaced-apart times of at least one quantitative biological parameter statistically related to biological parameter X, and optionally (ii) a value measured at a given time or at least two values measured at different spaced-apart times of at least one qualitative biological parameter statistically related to biological parameter X, for each subject in a cohort of subjects;
b) for each quantitative biological parameter, removing from the cohort or placing in a sub-group those subjects in whom this biological parameter shows drift or potential drift, and optionally for each qualitative biological parameter removing from the cohort or placing in a sub-group those subjects in whom this biological parameter is influential, to obtain an optimised cohort in particular from the values provided at step b);
c) optionally, providing the value measured at a given time, at least two values measured at different spaced-apart times and/or a mean of at least two values measured at different spaced-apart times of biological parameter X in the optimised cohort defined at step b).

The different terms are such as defined above in the different sections.

The optimised cohorts thus obtained may comprise for example a cohort of subjects in whom at least one given parameter does not show drift or potential drift, a cohort of subjects with possible drift of said at least one biological parameter and/or a cohort of subjects showing drift of said at least one biological parameter (e.g. drift and/or major drift).

Within the cohort(s) it is also possible to take into account different parameters influencing these parameters which show drift or potential drift.

The mean reference value and reference standard deviation are preferably defined according to the method for defining an optimised mean reference value and optimised reference standard deviation of a biological parameter X, such as defined above under the section having the same heading.

Evaluation of drift or potential drift of a biological parameter is notably carried out as described above in the method for analysing the metabolic drift of at least one biological parameter in a subject.

The method for optimising a cohort of subjects such as defined above is preferably implemented by computer or it is at least partly computer-implemented.

The method for optimising a cohort of subjects such as defined above is preferably an in vitro and/or ex vivo method.

The method for optimising a cohort of subjects such as defined above may therefore comprise a prior step to measure said biological parameter X and at least one biological parameter statistically related to said biological parameter, in a biological sample.

Between steps a) and b), the method may comprise a step to analyse the drift of at least one biological parameter and to infer therefrom whether this biological parameter is included in one of the three following states: no drift, having drift or having potential drift.

It is also possible to use sub-states.

For example, a state of no drift can be excellent or good.

For example, a drift state can be drift or major drift.

These states and/or sub-states are for example such as defined above.

It is possible to use sub-classes e.g. excellent good. At step b), (i), it is possible to define drift sub-classes e.g. potential drift, drift, major drift and out-of-tolerance. Biological parameters having «normal» values can be classified as excellent or good.

The method such as defined above may comprise subsequent steps to classify the results of the cohort study using the same method and study of changes in drift levels.

Method to Evaluate the Drift State of a Subject and/or to Assist Diagnosis of a Disease or Evaluation of a Subject's Risk of Suffering from a Disease

A further subject of the present invention is a method to evaluate the drift state of a subject and/or to assist diagnosis of a disease or evaluation of a subject's risk of suffering from a disease.

The method is preferably computer-implemented or at least partly computer-implemented.

The method is preferably an in vitro and/or ex vivo method.

In one preferred embodiment, this method comprises the implementing of a method for analysing the metabolic drift of at least one biological parameter in a subject, such as defined above under the section having the same heading.

In another preferred embodiment of the invention, the method to evaluate the drift state of a subject and/or to assist diagnosis of a disease or evaluation of a subject's risk of suffering from a disease comprises the following steps:
a) measuring the value of at least one biological parameter at least at one given time t1 in a biological sample of the subject, to obtain at least one value v1;
b) comparing the value(s) obtained at step a) with a reference threshold value and/or determining whether the values obtained at step a) fluctuate around a mean reference value; and
c) if the or one of the values of said biological parameter is higher than a maximum reference threshold value or lower than a minimum reference threshold value and/or if said values do not fluctuate round mean reference value, providing the value of at least one other biological parameter statistically related to said biological parameter and/or inferring therefrom whether the subject is suffering from is likely to suffer from the disease.

The method to evaluate the drift state of a subject and/or to assist diagnosis of a disease or evaluation of a subject's risk of suffering from a disease can comprise the following steps for example:
a) providing the value of at least one biological parameter measured at least at two different spaced-apart times t1 and t2 in a biological sample of the subject, to obtain at least two values v1 and v2;
b) determining whether the values obtained at step a) fluctuate around a mean reference value; and
c) if said values do not fluctuate around a mean reference value, providing the value of at least one other biological parameter statistically related to said biological parameter and/or inferring therefrom whether the subject is suffering from or likely to suffer from the disease.

The different terms are such as defined above.

In particular, fluctuation of the values around a mean reference value can be evaluated as defined above under the section «Method for analysing the metabolic drift of at least one biological parameter in a subject».

The mean reference value is preferably an optimised mean reference value such as defined above.

In the above methods, the quantitative biological parameter and/or said other statistically related biological parameter can be a direct or indirect marker of a disease or risk of suffering from a disease.

For example, the anti-thyroid peroxidase biological parameter is a marker of Hashimoto's thyroiditis and can also be related to a Graves-Basedow disease, or in smaller quantity it can be related to lupus, rheumatoid polyarthritis, hepatitis C or even breast cancer; the biological parameter showing the presence of the anti-Candida albicans antibody is a marker of infection with Candida Albicans; or the biological parameter showing the presence of an anti-Helicobacter pylori antibody is a marker of infection with Helicobacter Pylori.

In one preferred embodiment, the method such as defined above is characterized in that said biological parameter is the mean platelet volume, said statistically related biological parameter being free thyroxine and said disease being hypothyroidism and/or a Graves-Basedow disease.

For example, if the mean platelet volume shows low drift, the method may comprise measurement of free thyroxine (free T4). The probability of having an out-of-tolerance free thyroxine value is then higher than 30% in the population. If the mean platelet volume shows low drift and/or if free thyroxine T4 is effectively out-of-tolerance, it can be inferred that the subject is suffering from or is likely to suffer from hypothyroidism and/or Hashimoto's thyroiditis and/or a Graves-Basedow disease. The method may then comprise implementation of a diagnostic method, preferably in vitro, for hypothyroidism and/or Hashimoto's thyroiditis and/or Graves-Basedow disease.

In another embodiment, the method such as defined above is characterized in that said biological parameter is a multi-parameter indicator defined by the number of white blood cells divided by the number of red blood cells, and said disease is infection with Candida albicans, e.g. invasive candidiasis.

The number of white blood cells divided by the number of red blood cells is a much more significant indicator than the number of leukocytes or lymphocytes to estimate the risk of positive response to anti-Candida albicans antibodies and hence the risk of suffering from a disease related to infection with Candida albicans.

In another embodiment, the method such as defined above is characterized in that said biological parameter is a multi-parameter indicator defined by the number of lymphocytes divided by the number of polymorphonuclear neutrophils and said disease is infection with Cytomegalovirus (CMV).

The number of lymphocytes divided by the number of polymorphonuclear neutrophils is a more significant indicator for the detection of a positive response to anti-cytomegalovirus IgG antibodies than the number of lymphocytes alone for example, or the number of polymorphonuclear neutrophils alone. It is then possible to evaluate the drift of this indicator to further improve the accuracy of detection of infection with Cytomegalovirus.

Computer Programme

A further subject of the invention is a computer programme comprising programme code instructions to execute one or more steps of one of the methods such as defined above, when said programme is executed on a computer.

One subject of the present invention is therefore a computer programme comprising programme code instructions to execute one or more steps of the method for analysing the metabolic drift of at least one quantitative biological parameter in a subject, such as defined above, when said programme is executed on a computer in particular steps b) and c).

One subject of the present invention is therefore a computer programme comprising programme code instructions to execute one or more steps of the method for defining an optimised mean reference value and optimised reference standard deviation of a biological parameter X, such as defined above, when said programme is executed on a computer, in particular steps b), c) and d), or steps b), c), c′) and d).

One subject of the present invention is therefore a computer programme comprising programme code instructions to execute one or more of the steps of the method for optimising a cohort of subjects for the purpose of studying a biological parameter X, such as defined above, when said programme is executed on a computer, in particular step b).

One subject of the present invention is therefore a computer programme comprising programme code instructions to execute one or more steps of the method for evaluating drift state in a subject and/or to assist diagnosis of a disease or evaluation of the risk of suffering from a disease in a subject, such as defined above, when said programme is executed on a computer, in particular steps b) and c).

A further subject of the present invention is a computer-readable recording medium on which a computer programme is recorded comprising programme code instructions to execute one or more steps of one of the methods defined above, the computer programme preferably being such as defined above.

In one advantageous embodiment, the computer programme comprises programme code instructions to:
record other biological parameters as and when they are obtained;
finetune the optimised mean and standard deviation of each biological parameter;
carry out dynamic search for at least one other influential biological parameter; and/or
automatically edit a modifiable prescription.

Other characteristics and advantages of the invention will become better apparent from the following nonlimiting, illustrative examples.

### FIGURES

FIG. 1: Analysis of metabolic drift of leukocytes in a subject. Optimised mean: 5561.7/mm3; Optimised standard deviation: 1405.8/mm3; High reference threshold value: 10500/mm3; Low reference threshold value: 4000/mm3.

FIG. 2: Analysis of metabolic drift of MCV in a subject. Optimised mean: 87.1; Optimised standard deviation: 4.9 g/dl; High reference threshold value: 97 g/dl; Low reference threshold value: 82 g/dl.

FIG. 3: Analysis of metabolic drift of haematocrit in a subject. Optimised mean: 43.7%; Optimised standard deviation: 1.05%; High reference threshold value: 47%; Low reference threshold value: 37%.

FIG. 4: Analysis of metabolic drift of polymorphonuclear neutrophils in a subject. Optimised mean: 2931.9 mm3; Optimised standard deviation: 1016.8 mm3; Maximum reference threshold value: 7000 mm3; Minimum reference threshold value: 1500 mm3.

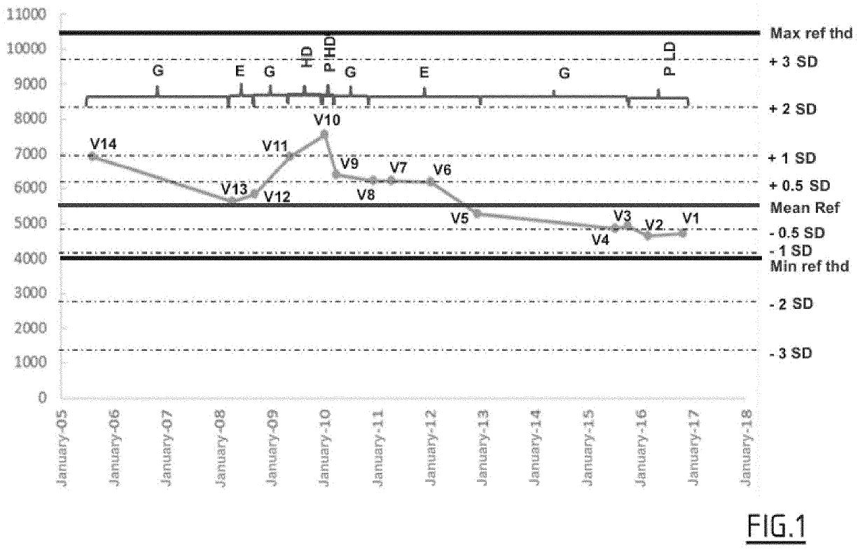

In FIGS. 1 to 4, «G» indicates a «Good» drift state; «E» indicates an «Excellent» drift state; «HD» indicates high drift; «LD» indicates low drift; «P LD» indicates potential low drift; «P HD» indicates potential high drift; «Maj LD» indicates major low drift; «SD» indicates «optimised reference standard deviation»; «Max ref thd»: maximum reference threshold defined by the French national authority for Health (Haute Autorité de Santé—HAS); «Min ref thd»: minimum reference threshold defined by HAS; «Mean ref» indicates optimised mean reference value.

### EXAMPLES

### Example 1: Analysis of Metabolic Drift of Vitamin B12

Material and Methods

(i) Determination of the Optimised Mean Reference Value and Optimised Reference Standard Deviation for Vitamin B12

The initial group of subjects comprised 67 subjects.

The examined biological parameters included inter alia vitamin B12, vitamin D, red cell count, MCV, haemoglobin level, leukocyte count, polymorphonuclear neutrophils, polymorphonuclear eosinophils, polymorphonuclear basophils, lymphocytes, monocytes, platelets, mean platelet volume, glucose (fasting), glycated haemoglobin, uric acid, creatinine, ASAT and ALAT, GGT, bilirubin, free bilirubin, conjugated bilirubin, HLA DQ, HLA A, HLA B, HLA C, HLA DR.

The value of the biological parameters in the initial group are given at time t.

The biological parameters identified as being statistically related to the concentration of vitamin B12 chiefly comprise vitamin D, HLA-DQ3, red cell count and haemoglobin level.

The vitamin D concentration was out-of-tolerance in more than 61 to 72% of patients showing low drift of vitamin B12. 37 subjects having a vitamin D concentration with drift, potential drift or out-of-tolerance were therefore removed from the group to calculate the mean reference value and reference standard deviation.

The mean and standard deviation were therefore calculated in the remaining group of 30 subjects.

(ii) Analysis of Metabolic Drift

The vitamin B12 values measured over time in this patient were the following: v1=250 pg/ml; v2=300 pg/ml; v3=278 pg/ml (measurements spaced apart by 3 months).

Value v1 is equal to 1.36 optimised reference standard deviation from the optimised mean reference value, value v2 is equal to 1.09 and value v3 is equal to 1.21.

The absolute value of the difference between the optimised mean reference value and the mean v1+v2 is equal to 225.5 pg/ml, i.e. equal to 1.22×optimised reference standard deviation.

The values v1 and v2 therefore show drift.

The absolute value of the difference between the optimised mean reference value and the mean v2+v3 is equal to 211.5 pg/ml, i.e. equal to 1.15×optimised reference standard deviation.

Values v2 and v3 therefore show drift.

On this account, it is also possible to examine the drift of the mean of v1, v2 and v3.

The absolute value of the difference between the optimised mean reference value and the mean v1+v2+v3 is equal to 224.5 pg/ml, i.e. equal to 1.22×optimised reference standard deviation.

Vitamin B12 therefore shows drift. On this account, vitamin D probably has drift and the level thereof is measured for verification.

Results

(1) Optimised Mean Reference Value and Optimised Reference Standard Deviation for Vitamin B12

The optimised mean reference value and optimised reference standard deviation of vitamin B12, calculated in particular in the sub-group not comprising subjects having drift, potential drift or out-of-tolerance concentrations of vitamin D, were respectively 500.5 pg/ml and 184.5 pg/ml.

(2) Analysis of Metabolic Drift of Vitamin B12 in a Subject

The optimised mean reference value and optimised reference standard deviation previously obtained were used to evaluate the metabolic drift of vitamin B12 in a subject.

The vitamin B12 values measured over time in this patient were: 250 pg/ml; 300 pg/ml; 278 pg/ml (measurements spaced apart by 3 months).

The range of reference values for vitamin B12 used in France is from 180 pg/ml to 914 pg/ml.

Using the optimised mean reference value ±2 optimised standard deviations as conventional tolerance thresholds, an optimised minimum threshold value of 131.5 and optimised maximum threshold value of 869.5 are obtained.

In both cases, it is ascertained that the values measured in this subject lie within the range of reference values according to methods currently used.

However, the measured values of vitamin B12 levels do not fluctuate around the optimised mean reference value. More specifically, the mean level of these three points lies at 1.2. standard deviation from the optimised mean reference value. Vitamin B12 therefore has drift which is not detected by methods currently used.

Since vitamin D is statistically related to vitamin B12, the vitamin D concentration in this subject is to be measured over time. Similarly, the values obtained will probably not fluctuate around the mean reference value and will show drift or potential drift.

The subject will then be given vitamin D treatment e.g. at a dose of 1 ampoule of 100 000 IU every 3 months and vitamin B12 for example via injection or oral route.

The subject's concentration of vitamin B12 and vitamin D are to be measured over time until sufficient correction is obtained.

### Example 2: Analysis of the Metabolic Drift of Different Biological Parameters in One Same Subject

FIG. 1 shows chronic drift of leukocytes for 1 year before returning to a balanced state. The measured values of the leukocytes are nevertheless starting to show low levels. This biological parameter must therefore be regularly checked.

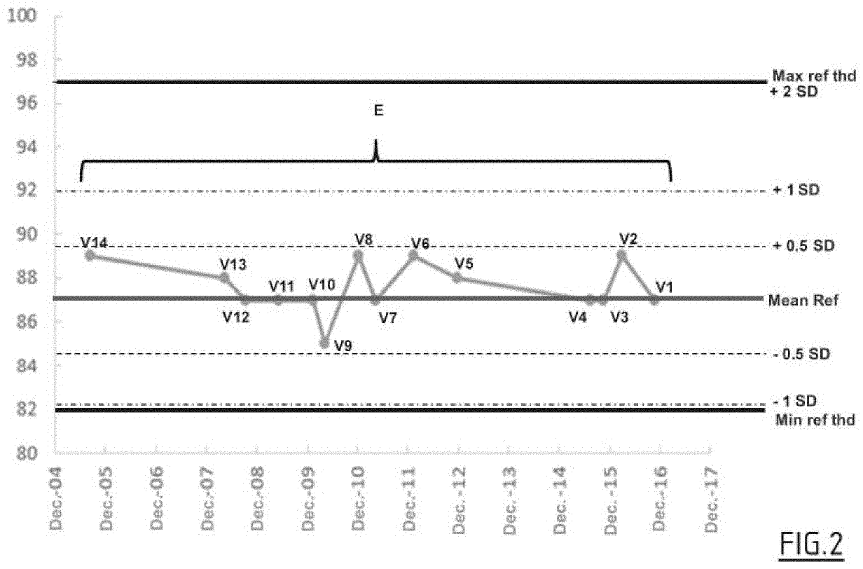

FIG. 2 concerns analysis of the metabolic drift of MCV. No drift of this biological parameter has been observed for 12 years. This demonstrates that a biological parameter can remain stable over time.

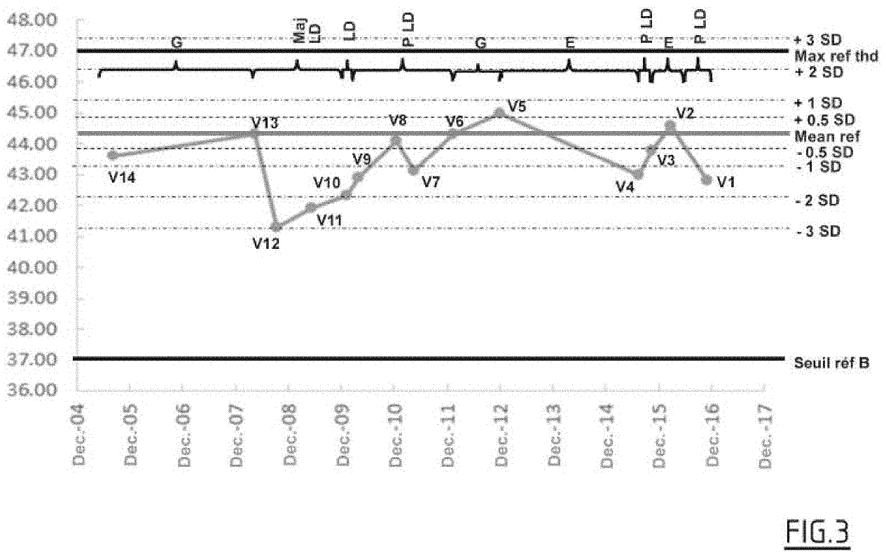

FIG. 3 shows major chronic drift of haematocrit for 18 months before returning to a stable state.

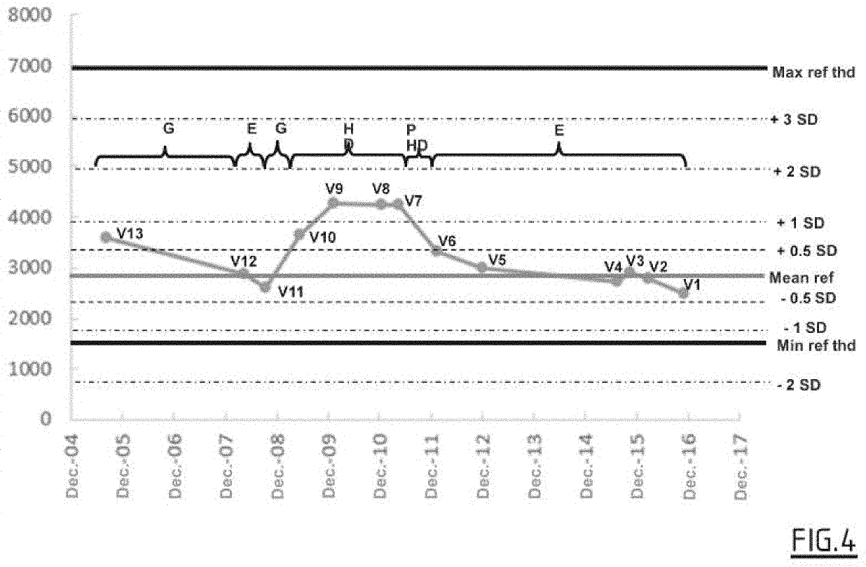

FIG. 4 shows chronic drift of polymorphonuclear neutrophils for 18 months before returning to an excellent state.

1. Method for analysing the metabolic drift of at least one quantitative biological parameter in a subject, characterized in that it comprises the following steps:
a) providing the values of at least one quantitative biological parameter measured at least at two different spaced-apart times t1 and t2, to obtain at least two values v1 and v2;
b) determining whether said values fluctuate around a mean reference value for said quantitative biological parameter;
c) if said values do not fluctuate around said mean reference value, concluding therefrom that said biological parameter shows drift or potential drift.

2. The analysis method according to claim 1, characterized in that the quantitative biological parameter measured at step a) is selected from the group consisting of a serum parameter, infection parameter, clinical parameter and multi-parameter indicator.

3. The analysis method according to claim 1, characterized in that:
said quantitative biological parameter shows drift if:
the absolute value of the difference between (i) the mean of said values and (ii) the mean reference value is higher than or equal to 1 reference standard deviation; or
the absolute value of the difference between (i) the mean of said values and (ii) the mean reference value is higher than or equal to 0.5 reference standard deviation and lower than 1 reference standard deviation, and the absolute value of the difference between (i) at least one of said values and (ii) the mean reference value is higher than or equal to 2 reference standard deviations; or
at least one of said values is higher than or equal to a maximum reference threshold value and/or at least one of said values is lower than or equal to a minimum reference threshold value; and/or
said quantitative biological parameter shows potential drift if:
the absolute value of the difference between (i) at least one of said values or the mean of said values and (ii) the mean reference value is higher than or equal to 0.5 reference standard deviation and lower than 1 reference standard deviation; and
if the absolute value of the difference between (i) each of said values and (ii) the mean reference value is lower than 2 reference standard deviations.

4. The analysis method according to claim 1, characterized in that when said quantitative biological parameter shows drift or potential drift, said method comprises a subsequent step to measure the value of said quantitative biological parameter at another time ti.

5. The analysis method according to claim 1, characterized in that when said quantitative biological parameter shows drift or potential drift, said method comprises at least one of the following subsequent steps:
providing the value of at least one other biological parameter statistically related to the biological parameter showing drift or potential drift; and/or
implementing a method to diagnose a disease or risk of suffering from a disease, said disease being associated with said biological parameter showing drift or potential drift or with a biological parameter statistically related to said biological parameter showing drift.

6. The analysis method according to claim 1, characterized in that the mean reference value and/or reference standard deviation are defined according to the method of claim 7.

7. Method for defining an optimised mean reference value and optimised reference standard deviation of a quantitative biological parameter X, comprising:
a) providing (i) a value of the quantitative biological parameter X measured at a given time or at least two values of the quantitative biological parameter X measured at different spaced-apart times and (ii) at least two values of at least one other quantitative biological parameter measured at different spaced-apart times, for each subject in a group of at least 50 subjects;
b) identifying the quantitative biological parameter(s) statistically related to the quantitative biological parameter X from one of said values and/or from the mean of said values of the quantitative biological parameter X, and from one of said values or the mean of said values of at least one other quantitative biological parameter;
c) for each biological parameter identified at step b), removing from the group those subjects in whom this biological parameter shows drift or potential drift, to define a sub-group of subjects;
c′) optionally, providing at least two values of at least one serum biological parameter measured at different spaced-apart times for each subject in the sub-group defined at step c) and removing from the sub-group those subjects in whom this serum biological parameter shows drift or potential drift, to define a second sub-group of subjects; and
d) defining the mean and standard deviation of the biological parameter X in the sub-group of subjects defined at step c) or in the second sub-group of subjects defined at step c′).

8. The method according to claim 7, characterized in that step a) further comprises providing the value of at least one other qualitative biological parameter measured at a given time and/or values of at least one other qualitative biological parameter measured at different spaced-apart times, in that step b) further comprises the identification of qualitative biological parameter(s) statistically related to the quantitative biological parameter X from one of said values and/or from the mean of said values of the quantitative biological parameter X and from said value(s) of at least one other qualitative biological parameter, and in that step c) further comprises the removal from the group of those subjects in whom this qualitative biological parameter is influential.

9. Method for evaluating the drift state of a subject and/or to assist diagnosis of a disease or evaluation of the risk of suffering from a disease in a subject, characterized in that it comprises implementation of the method for analysing metabolic drift in a subject according to claim 1.

10. Method for optimising a cohort of subjects for the purpose of studying a biological parameter X, comprising the steps of:
a) providing at least two values measured at different spaced-apart times and/or a mean of at least two values measured at different spaced-apart times of at least one quantitative biological parameter statistically related to biological parameter X, for each subject in a cohort of subjects;
b) for each quantitative biological parameter, removing from the cohort or placing in a sub-group those subjects in whom this biological parameter shows drift or potential drift, to obtain an optimised cohort;
c) optionally, providing the value at a given time, at least two values measured at different spaced-apart times and/or a mean of at least two values measured at different spaced-apart times of the biological parameter X in the optimised cohort defined at step b).

11. The method according to claim 10, characterized in that step a) further comprises the providing of a value measured at a given time or of at least two values measured at different spaced-apart times of at least one qualitative biological parameter statistically related to biological parameter X, for each subject in the cohort of subjects, and in that step b) for each qualitative biological parameter further comprises removing from the cohort or placing in a sub-group those subjects in whom this qualitative biological parameter is influential.

12. Method to assist diagnosis of a disease or evaluation of the risk of suffering from a disease in a subject, comprising:
a) measuring the value of at least one biological parameter at least at one time t1 in a biological sample of the subject, to obtain at least one value v1;
b) comparing the value(s) obtained at step a) with a reference threshold value and/or determining whether the values obtained at step a) fluctuate around a mean reference value; and
c) if the value or one of the values of said biological parameter is higher than a maximum reference threshold value or lower than a minimum reference threshold value and/or if said values do not fluctuate around a mean reference value, providing the value of at least one other biological parameter statistically related to said biological parameter and/or deducing therefrom that the subject is suffering from or likely to suffer from the disease,
characterized in that (i) said biological parameter is the mean platelet volume, said statistically related biological parameter is free thyroxine and said disease is hypothyroidism and/or a Graves-Basedow disease or (ii) said biological parameter is a multi-parameter indicator defined by the number of white blood cells divided by the number of red blood cells and said disease is infection with Candida albicans, preferably invasive candidiasis, or (iii) said biological parameter is a multi-parameter indicator defined by the number of lymphocytes divided by the number of polymorphonuclear neutrophils and said disease is infection with Cytomegalovirus.

13. Computer programme comprising programme code instructions to execute the steps of a method according to claim 1, when said programme is executed on a computer.

14. Computer programme comprising programme code instructions to execute the steps of a method according to claim 7, when said programme is executed on a computer.

15. Computer programme comprising programme code instructions to execute the steps of a method according to claim 9, when said programme is executed on a computer.

16. Computer programme comprising programme code instructions to execute the steps of a method according to claim 10, when said programme is executed on a computer.

17. Computer programme comprising programme code instructions to execute the steps of a method according to claim 12, when said programme is executed on a computer.
